(19) United States
(12) Patent Application Publication
(10) Pub. No.: US 2021/0394202 A1
(43) Pub. Date: Dec. 23, 2021

(54) IONIZERS HAVING CARBON NANOTUBE ION EMITTING HEADS

(71) Applicant: Headwaters, Inc., Marblehead, MA (US)

(72) Inventors: Troy G. Anderson, Marblehead, MA (US); Thomas A. Rounds, East Greenwich, RI (US); Andres A. Arrocha-Arcos, Cuernavaca (MX)

(21) Appl. No.: 17/351,847

(22) Filed: Jun. 18, 2021

Related U.S. Application Data
(60) Provisional application No. 63/041,326, filed on Jun. 19, 2020.

(51) Int. Cl. B03C 3/60; B03C 3/38; B03C 3/41; B03C 3/32; A61M 16/10

(52) U.S. Cl. CPC B03C 3/60; B03C 3/38; B03C 3/41; A61M 2209/088; A61M 16/10; B03C 2201/04; B03C 3/32

(57) ABSTRACT

Ionizers having carbon nanotube (CNT) ion emitting heads of the present invention include ionization circuitry supplying voltage at ionization potential and carbon nanotube (CNT) ion emitters operatively connected thereto. The carbon nanotube (CNT) ion emitters are mechanically and electrically stable, provide good ion flows for air purification and/or air invigoration when actuated at ionization potential and are capable of prolonged use in highly polluted environments.

### CROSS-REFERENCE TO RELATED APPLICATIONS

This application is related to U.S. Pat. No. 7,215,526, entitled Ion Generator with Open Emitter and Safety Feature, issued May 8, 2007 to Constantinos; to U.S. Pat. No. 7,595,030, entitled. Air-circulating Ionizing Air Cleaner, issued Sep. 29, 2009 to Joannou; to U.S. Pat. No. 6,919,053, entitled Portable Ion Generator and Dust Collector, issued Jul. 19, 2005 to Joannou; to U.S. Pat. No. 9,737,895, entitled Personal Rechargeable Portable ionic Air Purifier, issued Aug. 22, 2017 to Genereux; and to Vanderbelt, allowed U.S. utility application Ser. No. 15/914,036, entitled Personal Rechargeable Portable Ionic Air Purifier, filed Mar. 7, 2018; each incorporated herein by reference in their entirety.

### FIELD OF THE INVENTION

The invention relates to ion generators, more particularly, to battery-operated portable ion generators for personal use and for air purification and cleansing, and still more particularly, to such ionizers having carbon nanotube (CNT) ion emitting heads.

### BACKGROUND OF THE INVENTION

Portable wearable and other ionizers controllably emit ions into the air which entrain particulates and remove the same from the environment of the user. The particulates may be dirt, dust, viruses, particles of bacteria and the like. They also emit, among other things, negative ions (NAIS) into the air space of the ionizers, which have well-known beneficial effects, including to relieve symptoms of some allergies, and to improve mental health and overall well-being.

The heretofore known ion emitting heads of these ionizers typically have included icon emitters that have been made of metal pin or carbon fiber brush electrodes.

Strands of carbon fiber are currently used to emit ions. High voltage supplies provide current to these carbon fiber strands and the carbon fiber strands release ions into the air whereby the ions attach themselves to airborne particles and change the polarity of the airborne particles. The charged particles are then attracted to oppositely charged surfaces or to ground thereby cleaning the air of such particles with the result that the particles do not enter the human respiratory system. Such carbon fiber brushes have been used for ion emission because they are effective ion producers but do not emit as much ozone as metal pin emitters.

The personal ionic air purifiers of Genereux, U.S. Pat. No. 9,737,895, entitled Personal Rechargeable Portable Ionic Air Purifier, issued Aug. 22, 2017, for example, use carbon fiber brushes to controllably emit negative ions.

Emitters with broken or worn carbon fibers negatively affect ion generating performance: (I) the carbon fibers are fragile—they break off fairly easily with impact or abrasion, and (ii) they are prone to carbon fiber brush erosion (shortening) of the fibers if the supplied voltage is too high or if environmental factors such as high humidity or high airborne smog levels are present. Moreover, (iii) they typically do not allow for higher levels of voltage that are required for increasingly higher levels of ion output.

### SUMMARY OF THE INVENTION

It accordingly is the general object of the present invention to disclose ionizers not subject to the disadvantages and disutilities of the heretofore known ionizers having carbon fiber bristle or metal pin ion emitters.

It is a related object of the present invention to provide the same or greater ion flow than the heretofore known carbon fiber bristle ion emitters of personal or other ionizers for air cleansing and purification.

It is another related object of the present invention to provide reduced breakage than the heretofore known carbon fiber bristle ion emitters of such ionizers from impact, for example, as from clothing rubbing against such bristles.

It is a further related object of the present invention to provide reduced wear/erosion (burning-off or “shortening”) than the heretofore known carbon fiber bristle ion emitters of such ionizers during use.

It is another related object of the present invention to increase the useful life than that of the heretofore known carbon fiber bristle ion emitters despite prolonged use in polluted environments or when operated at comparatively-high voltages.

In accord with these and other objects, the present invention discloses personal, wearable and other air purifying ionizers having carbon nanotube (CNT) ion emitting heads. The carbon nanotube (CNT) ion emitting heads of the present invention are mechanically stable, and thereby reduce if not eliminate unwanted breakage, and are electrically stable, and thereby reduce if not eliminate electrode erosion, even if the ionization voltage supplied thereto is high or if there is a high concentration of air pollutants.

The air purifying ionizers of the present invention include a housing. The housing is a wearable housing in one disclosed embodiment. A carbon nanotube (CNT) ion emitting head is mounted to the housing. In one embodiment, the carbon nanotube (CNT) ion emitting head is comprised of a plurality of carbon nanotube (CNT) filaments arranged like a brush and in another embodiment it is comprised of a plurality of elongated carbon nanotube (CNT) filaments braided like a rope. An ionization circuit supplying a voltage to the carbon nanotube (CNT) ion emitting head at ionization potential is mounted in the housing.

In one disclosed carbon nanotube (CNT) ion emitting head embodiment, at least two (2) spaced-apart carbon nanotube (CNT) ion emitters are arrayed in-line that increase the flow of ions in dependence on the number of carbon nanotube (CNT) ion emitters arrayed and in another carbon nanotube (CNT) ion emitting head embodiment at least three (3) spaced-apart carbon nanotube (CNT) ion emitters are arrayed in a U-shape to selectively steer the flow of ions towards a user's respiratory system.

It is a further object of the present invention to disclose carbon nanotube (CNT) ion emitting heads that may be used as new or replacement heads instead of the heretofore known carbon fiber bristle ion emitters of the heretofore known personal and other air purifying and/or invigorating ionizers.

In accord therewith, the present invention discloses carbon nanotube (CNT) ion emitting heads for ion generating air purifiers including a housing and an ionization circuit including a high-voltage step-up transformer and having an output that supplies voltage at ionization potential mounted in the housing, comprising an elongated, electrically conductive interconnection means having ends a carbon nanotube (CNT) ion emitter provided on one end of said elongated, electrically conductive interconnection means; and a plug mounted to the other end thereof that is adapted to electrically connect with said output of said ionization control circuit supplying voltage at ionization potential; whereby, when said plug is connected to said output of said ionization control circuit, said voltage energizes said carbon nanotube (CNT) ion emitter causing it to emit ions.

It is another object of the present invention to provide improved yields of ion emission from the ionizers having carbon nanotube (CNT) ion emitting heads of the present invention.

In accord therewith, to increase surface area and thereby yield higher ion emission, a coat of a preselected chemical such as a one (1) molar solution of HNO3 (nitric acid) potentially boosted with one percent (1%) to two percent (2%) H2O2 is advantageously applied to any carbon nanotube (cNT) ion emitter and any ionizer of the present invention.

### BRIEF DESCRIPTION OF THE DRAWINGS

These and other objects, advantageous features and inventive aspects of the present invention will become apparent as the invention becomes better understood by reference to the following detailed description of the presently preferred embodiments thereof, and to the drawings, wherein:

FIG. 1A is a pictorial view of a prior art portable rechargeable personal ionic air purifier having a carbon fiber bristle emitter and FIG. 1B is a broken away pictorial view thereof illustrating the prior art carbon fiber bristle emitter reduced to a stub due to mechanical breakage and/or electrical consumption;

FIG. 2A is a pictorial diagram schematically illustrating a carbon nanotube (CNT) ion emitter of elongated carbon nanotube (CNT) filaments arranged side-by-side like a brush and FIG. 2B is a pictorial diagram schematically illustrating a carbon nanotube (CNT) emitter of carbon nanotube (CNT) filaments braided like a rope;

FIG. 3A is a pictorial diagram schematically illustrating a carbon nanotube (CNT) ion emitter linear array and FIG. 3B is a pictorial diagram schematically illustrating a carbon nanotube (CNT) ion emitter U-shaped array;

FIG. 4 is a pictorial diagram schematically illustrating an ionizing air purifier having a carbon nanotube (CNT) ion emitting head in accord with the present invention;

FIG. 5 is a pictorial diagram schematically illustrating the manner that carbon nanotube (CNT) ion emitting heads of the present invention may be connected to ionization circuitry to retrofit existing or newly fashioned ionizing air purifiers with the carbon nanotube (CNT) ion emitting heads of the present invention; and

FIG. 6 is a pictorial diagram schematically illustrating one presently preferred embodiment of a carbon nanotube (CNT) ion emitting head in accord with the present invention.

### DETAILED DESCRIPTION OF THE PRESENTLY PREFERRED EMBODIMENTS

It is to be understood that the invention is not limited in its application to the details of construction or to the arrangements of the components set forth in the following description or illustrated in the drawings. The invention is capable of other embodiments of being practiced and carried out in various ways. Also, it is to be understood that the phraseology and terminology employed herein are for the purpose of the description and should not be regarded as limiting.

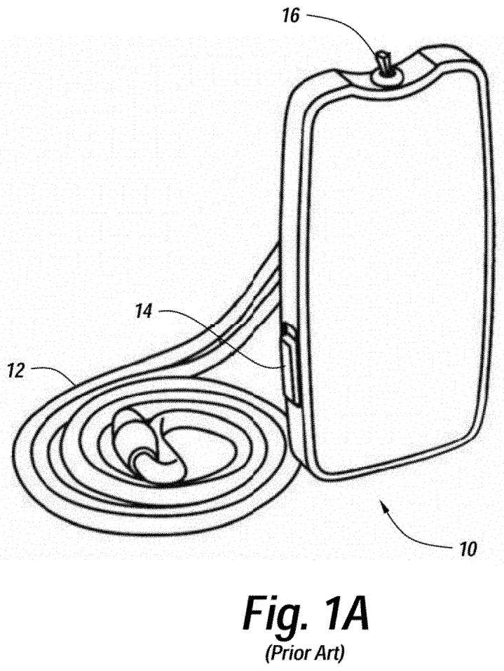

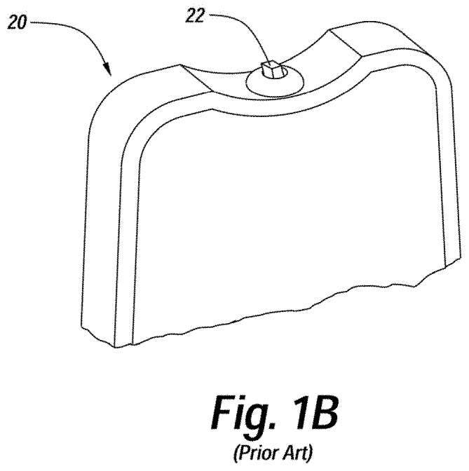

Turning now descriptively to the drawings, FIG. 1A illustrates generally at 10 a prior art portable, rechargeable, personal ionic air purifier. The user hangs the purifier 10 around their neck using the conductive cord or lanyard 12 and turns it “on” using the switch 14. A cloud of ions, not shown, is thereby produced off of carbon fiber brush ion emitter 16 towards the facial area, not shown, to energize the personal airspace with ions and remove particulates therefrom. These ions attract opposite charged particles in the air and are then attracted together towards the nearest ground source. The conductive cord 12 ensures that the ground source is the body of the user and not the breathable air stream, thus effectively cleaning the breathable air stream of contaminants in the air and/or producing invigorating negative ions (NAIS). However, ion emitters with broken or worn carbon fibers adversely impact ion generating performance and therewith their air cleaning and/or air invigorating capacity.

Referring now to FIG. 1B, generally designated at 20 is the prior art portable, rechargeable, personal ionic air purifier of FIG. 1A, where the carbon fiber bristle emitter thereof is shown reduced to a mere stub 22, due to, among other things, mechanical breakage, electrical consumption, high use in such environments as those having high humidity or high airborne smog levels, and/or due to comparatively-high supplied voltage levels, thereby necessitating the replacement of the entire air purifier unit itself or of the degraded carbon fiber bristle ion emitter head thereof, with the consequent attendant expense, service interruption and trouble.

Ion emitting elements of the personal or other ionizers having carbon nanotube (CNT) ion emitting heads of the present invention are constructed from carbon nanotube (CNT) material that exhibits the requisite electrical conductivity and electrical stability and has sufficient tensile strength to provide purifying and/or invigorating ion flows, both reducing if not eliminating electrode breakage and reducing if not eliminating electrode erosion, even if the ionization voltage is comparatively-high or if there is a comparatively-high concentration of environmental air pollutants.

The portable wearable ionizing air purifiers and other ionizers of the present invention, due to the mechanical strength, electrical stability and ion generating capacity of their carbon nanotube (CNT) ion emitters, provide a long and useful operational life while purifying the breathable air of pollutants and/or simultaneously emitting beneficial negative ions (NAIS) into the breathable airspace.

Carbon nanotubes (CNTs) are hollow tubes made of carbon with diameters measured in nanometers and may be fashioned in any manner well known to those of skill in the art. Any carbon nanotube (CNT) material of construction or design that provides comparatively-high ion emission, promotes or provides comparatively-long ion emitter durability and/or comparatively-long ion emitter lifetimes may be employed without departing from the inventive concepts. It is believed that the narrower their diameter, the sharper the tips, the greater the ion emission.

A carbon nanotube (CNT) ion emitting head of the present invention can in different embodiments have carbon nanotube (CNT) filaments loosely arranged like a bristle brush or braided like a rope or otherwise arranged, constituted and fashioned without departing from the inventive concepts.

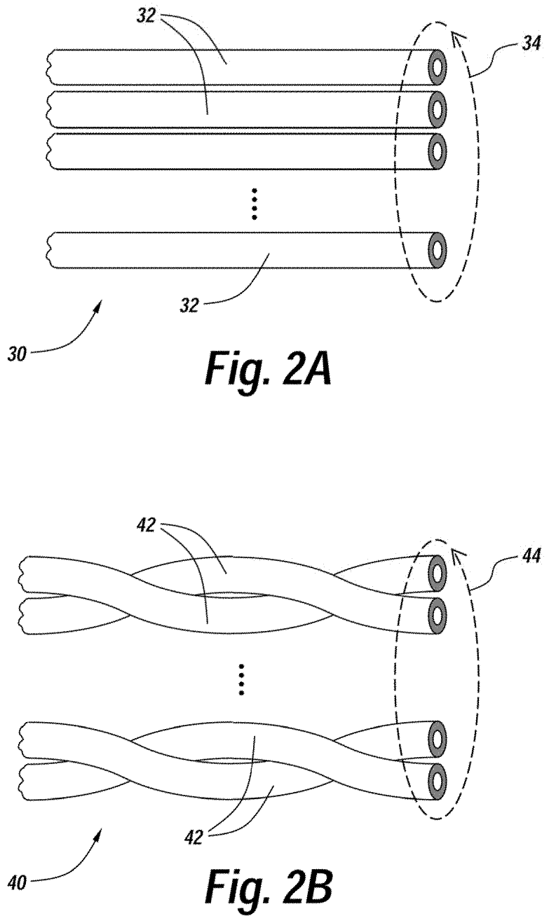

Referring now to FIGS. 2A, 2B, generally designated at 30 in FIG. 2A and at 40 in FIG. 2B are pictorial diagrams schematically illustrating different carbon nanotube (CNT) ion emitting head embodiments in accord with the present invention.

The carbon nanotube (CNT) ion emitting head 30 of the FIG. 2A includes a plurality of carbon nanotube (CNT) filaments 32 bundled together side-by-side like a brush, schematically illustrated in dashed outline 34.

The carbon nanotube (CNT) ion emitting head 40 of the FIG. 2B includes a plurality of braided carbon nanotube (CNT) filaments 42 that are bundled together like a braided rope, schematically illustrated in dashed outline 44.

Carbon nanotube (CNT) material other than carbon nanotube (CNT) filaments, such as carbon nanotube (CNT) ribbons, fabric or tape, may be employed without departing from the inventive concepts.

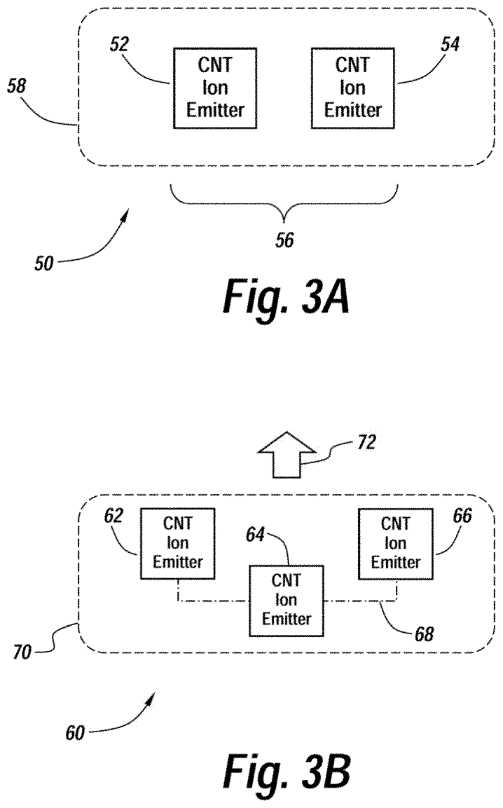

Referring now to FIGS. 3A, 3B, generally designated at 50 in the FIG. 3A and at 60 in the FIG. 3B are pictorial diagrams schematically illustrating different carbon nanotube (CNT) ion emitting head array embodiments in accord with the present invention.

The carbon nanotube (CNT) ion emitting head array 50 of the FIG. 3A includes a linear array of two (2) carbon nanotube (CNT) ion emitters 52, 54 arranged in-line as schematically illustrated by bracket 56 on a housing schematically illustrated by dashed box 58. Although the array 50 as shown has, the two (2) carbon nanotube (CNT) ion emitters 52, 54 mounted in-line on the housing 58, it will be appreciated that more than two (2) carbon nanotube (CNT) ion emitters may be arranged in-line where ion output, and thus air cleaning performance and/or air invigoration, would be increased in dependence on the number of carbon nanotube (CNT) ion emitters arrayed.

The carbon nanotube (CNT) ion emitting head array 60 of FIG. 3B includes three (3) or more carbon nanotube (CNT) ion emitters 62, 64, 66 arranged in a U-shape, schematically illustrated by dashed line 68, that are mounted to a housing, schematically illustrated by dashed box 70.

The U-shaped carbon nanotube (CNT) ion emitting head array 60 is a directional array that targets ions at the user's head as schematically illustrated by arrow 72. Three (3) or more carbon nanotube (CNT) ion emitters may be otherwise arrayed to vector the ion stream 72 to improve protection of the wearer's respiratory system from airborne pollutants and/or to controllably direct the supply of invigorating negative ions (NAB) thereto without departing from the inventive concepts,

Referring now to FIG. 4, generally designated at 80 is a pictorial diagram schematically illustrating an air purifying ionizer in accord with the present invention. The ionizer 80 includes at least One carbon nanotube (CNT) ion emitter 82 that is operatively connected to an ionization circuit 84 that supplies it with voltage at or above ionization potential The at least one carbon nanotube (CNT) ion emitter 82 and ionization circuit 84, along with power and other circuitry and components, not shown, are mounted to a portable or other housing schematically illustrated by dashed box 86, such as the ionization circuit and housing of the aforesaid and incorporated by reference US patent to Genereux, although other ionization control circuitry and housings well-known to those of skill in the art such as those of any of the other aforesaid and incorporated by reference related United States patents and US patent application may be employed without departing from the inventive concepts.

The carbon nanotube (CNT) ion emitter 82 of the ionizer 80, when actuated at or above ionization potential, provides a flow of cleansing and/or invigorating ions that ionize the air surrounding the housing 86 and remove pollutants entrained thereon from the breathable airspace.

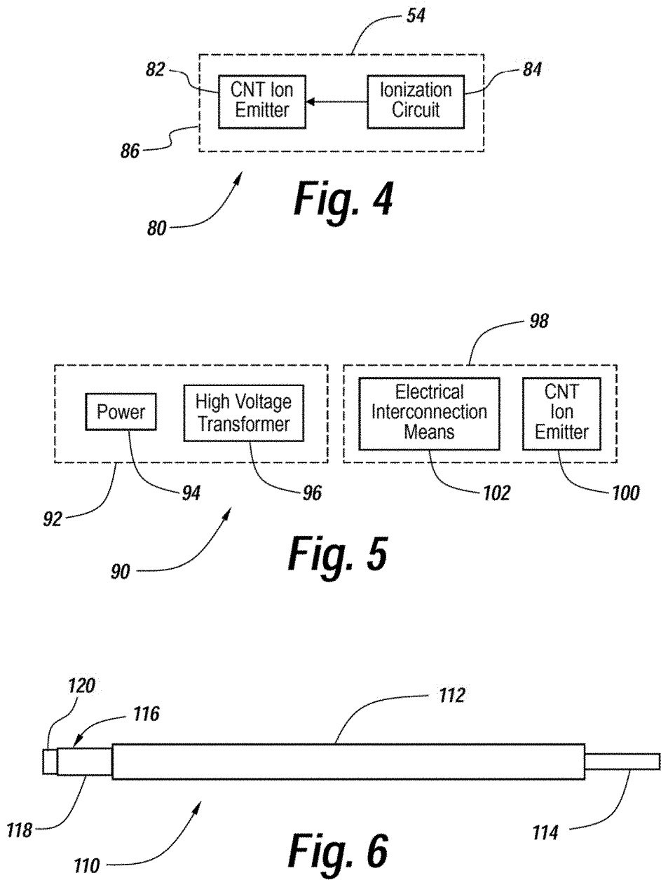

Referring now to FIG. 5, generally designated at 90 is a pictorial diagram schematically illustrating the manner that carbon nanotube (CNT) ion emitting heads of the present invention may be connected to ionization circuitry to retrofit already existing inventory or newly fashioned ionizing air purifiers with the improved carbon nanotube (CNT) ion emitters of the present invention. A personal rechargeable portable ionic air purifier is schematically illustrated by dashed box 92. The ionizer 92 is that of the aforesaid U.S. Pat. No. 9,737,895 although other ionizers such as those of any one of the aforesaid and incorporated by reference related United States patents and US patent application that include a source of power 94 and a high-voltage power circuit 96 mounted in a housing, not shown, may be employed without departing from the inventive concepts.

The output of the high-voltage power circuit 96 including the high-voltage step-up transformer provides voltage at ionization potential.

A carbon nanotube (CNT) ion emitting head schematically illustrated by dashed box 98 includes a carbon nanotube (CNT) ion emitter 100 and an elongated, conductive electrical interconnection means 102. The elongated, conductive electrical interconnection means 102 is adapted to electrically interconnect the output supplying voltage at ionization potential of the high voltage power circuit 96 of the ionic air purifier 92 to the carbon nanotube (CNT) ion emitter 100 of the carbon nanotube (CNT) ion emitting head 98, thereby energizing the carbon nanotube (CNT) ion emitter 100 and inducing it to emit cleansing and/or invigorating ions.

As will be readily appreciated by those of skill in the art, the carbon nanotube (cNT) ion emitting head 98 can be variously embodied, it may be replaceable or non-replaceable on the housing that receives it, its carbon nanotube (CNT) ion emitter 100 may be singly or multiply constituted, its carbon nanotube (CNT) ion emitter 100 may include filaments bristled like a brush or braided like a rope or otherwise fashioned or constituted; and its elongated, conductive electrical interconnection means 102 may be variously embodied, whether it is integrally formed, or comprised of components interoperable to provide its intended electrical interconnection functionality, while in each case adapted to electrically connect the carbon nanotube (CNT) ion emitter 100 to the output supplying voltage at ionization potential of the high voltage power circuit of the ionizer 92 to which the head 98 may be retrofit or for which it is newly fashioned such as those of any one of the aforesaid and incorporated by reference related United States patents and US patent application or other ionizers without departing from the inventive concepts.

Referring now to FIG. 6, generally designated at 110 is a pictorial diagram schematically illustrating a carbon nanotube (CNT) ion emitting head in accord with one presently preferred embodiment of the present invention. The carbon nanotube (CNT) ion emitting head 110 is used as a new or a replacement head for the personal rechargeable portable ionic air purifier as shown and described in the aforesaid U.S. Pat. No. 9,737,895.

The carbon nanotube (CNT) ion emitting head 110 includes an insulated and elongated, electrically conductive length of carbon nanotube (CNT) material 112. A portion of its insulative sheath is cut-away to expose a carbon nanotube (CNT) bristle ion emitter 114 at one end of the insulated and elongated, electrically conductive carbon nanotube (CNT) material 112.

In the presently preferred and illustrated embodiment, the carbon nanotube (CNT) bristle ion emitter 114 of the carbon nanotube (CNT) material 112 is comprised by a plurality of carbon nanotube (CNT) filaments, characterized by two hundred seventy (270) mg/m and one and twelve-one-hundreds (1.12) ohm/m, commercially available from DEXMAT corporation; in another embodiment, it is comprised by a plurality of carbon nanotube (CNT) filaments characterized by three hundred ninety (390) mg/m and sixty-eight-one-hundreds (0.68) ohm/m, also commercially available from DEXMAT corporation.

A female-type plug generally designated 116 is mounted to the other of the ends of the insulated and elongated, electrically conductive carbon nanotube (CNT) material 112 that is adapted to electrically connect with the output of the high-voltage power circuit supplying voltage at ionization potential, not shown, of the aforesaid U.S. Pat. No. 9,737,895. The female-type plug has a first U-shaped portion schematically illustrated at 118 that is mechanically crimped to an exposed end portion of CNT material 112 from which the insulative sheath has been stripped and a second U-shaped portion schematically illustrated at 120 electrically and mechanically joined to the first U-shaped. portion 118 that receives the output wire, not shown, supplying voltage thereto at ionization potential of the personal rechargeable portable ionic air purifier as shown and described in the aforesaid U.S. Pat. No. 9,737,895.

In further accord with the present invention, to increase surface area and thus to increase ion output, the carbon nanotube (CNT) ion emitter of any ionizer having a carbon nanotube (CNT) emitting bead of the present invention may be dipped in a chemical solution such as a one (1) molar solution of HNO3 (nitric acid), potentially boosted with one (1) to (2) percent (1%-2%) H2O2, although any other suitable coating that increases surface area and thereby ion emission could be employed without departing from the inventive concepts.

Many modifications of the presently disclosed invention will become apparent to those of skill in the art having benefit of the present disclosure without departing from the inventive concepts.

What is claimed is:

1. An air purifying ionizer, comprising:
a housing;
a carbon nanotube (CNT) ion emitter mounted to the housing and exteriorly extending therefrom into the air; and
an ionization circuit mounted in the housing supplying a voltage to the carbon nanotube (CNT) ion emitter at ionization potential inducing thereby the emission of ions from said exteriorly extending carbon nanotube (CNT) ion emitter into the air.

2. The air purifying ionizer of claim 1, wherein the (CNT) ion emitter is comprised of a plurality of carbon nanotube (CNT) filaments arranged like a brush.

3. The air purifying ionizer of claim 1, wherein the (CNT) ion emitter is comprised of a plurality of carbon nanotube (CNT) filaments braided like a rope.

4. The air purifying ionizer of claim 1, wherein said housing is a portable housing.

5. The air purifying ionizer of claim 4, wherein said portable housing is a wearable housing.

6. The air purifying ionizer of claim 1, wherein said carbon nanotube (CNT) ion emitter mounted to the housing includes first and second spaced-apart carbon nanotube (CNT) ion emitters arrayed in-line on said housing.

7. The air purifying ionizer of claim 1, wherein said carbon nanotube ion emitter mounted to said housing includes first, second and third spaced-apart carbon nanotube (CNT) ion emitters arrayed on said housing in a predetermined pattern determined to selectively direct ion emission anisotropically into the air surrounding said housing.

8. The air purifying ionizer of claim 7, wherein said pattern of said first, second and third spaced-apart carbon nanotube (CNT) ion emitters is a U-shaped pattern.

9. The air purifying ionizer of claim 7, wherein said housing is a wearable housing worn about the neck and said predetermined pattern determined to selectively direct ion emission anisotropically into the air surrounding said housing is determined to anisotropically vector ion emission towards the head of the wearer of the portable housing.

10. The air purifying ionizer of claim 1, further including a coat of a one (1) molar solution of HNO3 (nitric acid) on said carbon nanotube (CNT) ion emitter to increase its surface area and thereby yield higher ion emission therefrom.

11. The air purifying ionizer of claim 10, wherein said coat of a one (1) molar solution of HNO3 (nitric acid) is boosted with one (1) to two (2) percent (1% -2%) H2O2.

12. A carbon nanotube (CNT) ion emitting head for use as a replacement or newly fashioned head for an ion generating air purifier of the type having a housing and mounted therein an ionization control circuit having an output that supplies voltage at ionization potential, comprising:
an elongated, electrically conductive interconnection means having ends;
a carbon nanotube (CNT) ion emitter provided on one end of said elongated interconnection means; and
a plug mounted to the other end thereof that is adapted to electrically connect with said output of said ionization control circuit supplying voltage at ionization potential; whereby, when said plug is connected to said output of said ionization control circuit, said voltage energizes said carbon nanotube (CNT) ion emitter causing it to emit ions.

13. The carbon nanotube (CNT) ion emitting head of claim 12, wherein said carbon nanotube (CNT) ion emitter is a carbon nanotube (CNT) bristle ion emitter.

14. The carbon nanotube (CNT) ion emitting head of claim 12, wherein said carbon nanotube (CNT) ion emitter is a carbon nanotube (CNT) braided ion emitter.

15. The carbon nanotube (CNT) ion emitting head of claim 12, wherein said elongated, electrically conductive interconnection means includes an insulated, elongated, electrically conductive length of carbon nanotube (CNT) material having ends partially stripped of insulation at one end to provide said carbon nanotube (CNT) ion emitter.

16. The carbon nanotube (CNT) ion emitting head of claim 15, wherein said insulated, elongated, electrically conductive length of carbon nanotube (CNT) material having ends is partially stripped of insulation at its other end and said plug is a female-type plug mounted thereto.

17. The carbon nanotube (CNT) ion emitting head of claim 12, further including a coat of a one (1) molar solution of HNO3 (nitric acid) on said carbon nanotube (CNT) ion emitter to increase its surface area and thereby yield higher ion emission therefrom.

18. The carbon nanotube (CNT) ion emitting head of claim 17, wherein said coat of a one (1) molar solution of HNO3 (nitric acid) is boosted with one (1) to two (2) percent (1%-2%) H2O2.
